(19) United States
(12) Patent Application Publication
(10) Pub. No.: US 2026/0088159 A1
(43) Pub. Date: Mar. 26, 2026

(54) SYSTEM FOR UPDATING A MEDICAL FACILITY

(71) Applicant: KONINKLIJKE PHILIPS N.V., EINDHOVEN (NL)

(72) Inventors: MOUMITA SAHA, BANGALORE (IN); KRISHNAMOORTHY PALANISAMY, BANGALORE (IN)

(21) Appl. No.: 19/109,155

(22) PCT Filed: Sep. 4, 2023
(86) PCT No.: PCT/EP2023/074100

§ 371 Date: Mar. 6, 2025

Related U.S. Application Data
(60) Provisional application No. 63/406,771, filed on Sep. 15, 2022.

(51) Int. Cl. G16H 40/20; G16H 40/40

(52) U.S. Cl. CPC G16H 40/20; G16H 40/40

(57) ABSTRACT

A system (100A) for updating a medical facility includes a computer, an interface (140) to a communications network (103), and a system memory (102). The computer includes a computer memory (151) that stores instructions and a processor (152) that executes the instructions. The system memory (102) receives and stores information obtained from the medical facility via the interface (140). Based on the processor (152) executing the instructions, the computer is configured to: retrieve, from the system memory (102), the information obtained from the medical facility; apply an artificial intelligence model to the information obtained from the medical facility to determine whether to update the medical facility; and initiating at least one of maintenance or upgrading for the medical facility based on determining to update the medical facility.

### BACKGROUND

An intensive care unit (ICU) is an organized structure which provides specialized medical and nursing care for critically ill patients. The ICU may have various items of advanced equipment for monitoring, as well as for supporting multiple modalities of physiologic organ to sustain life for patients with severe or life-threatening illnesses and injuries. The ICU may provide care continuously from specially trained teams of staff and specialists. Various categories of ICUs exist for specialized functioning and operational support. The advanced equipment included in ICUs may consist of vital devices such as cardiac monitors to track critical signs, suction machines, mechanical ventilator to support respiration, infusion pumps to regulate the flow of medication, oxygen support, and other machines such as BiPAP & CPAP for respiratory support. ICUs are designed for critical and sensitive patients whose health conditions are completely monitored and supported by the sophisticated machines and devices. Thus, it is important to have a keen vigilance on the operational status of the items of equipment in ICUs. It is also necessary to monitor the comprehensive status of the ICUs and suggest appropriate upgrades on time-to-time basis based on need and utility.

### SUMMARY

According to an aspect of the present disclosure, system for updating a medical facility includes a computer, an interface to a communications network, and a system memory. The computer includes a computer memory that stores instructions and a processor that executes the instructions. The system memory receives and stores information obtained from the medical facility via the interface. Based on the processor executing the instructions, the computer is configured to: retrieve, from the system memory, the information obtained from the medical facility; apply an artificial intelligence model to the information obtained from the medical facility to determine whether to update the medical facility; and initiate at least one of maintenance or upgrading for the medical facility based on determining to update the medical facility.

According to another aspect of the present disclosure, a method for updating a medical facility includes receiving and storing, at a system memory, information obtained from the medical facility via an interface; retrieving, from the system memory by a system computer comprising a computer memory that stores instructions and a processor that executes the instructions, the information obtained from the medical facility; applying, by the system computer, an artificial intelligence model to the information obtained from the medical facility to determine whether to update the medical facility; and initiating, by the system computer, at least one of maintenance or upgrading for the medical facility based on determining to update the medical facility.

According to another aspect of the present disclosure, a computer for updating a medical facility includes a computer memory that stores instructions; and a processor that executes the instructions. Based on the processor executing the instructions, the computer is configured to: retrieve, from a system memory a system memory that receives and stores information obtained from the medical facility via an interface, the information obtained from the medical facility; apply an artificial intelligence model to the information obtained from the medical facility to determine whether to update the medical facility; and initiate at least one of maintenance or upgrading for the medical facility based on determining to update the medical facility.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for updating a medical facility, in accordance with a representative embodiment.

FIG. 1B illustrates another system for updating a medical facility, in accordance with a representative embodiment.

FIG. 2 illustrates an AI-based system for updating a medical facility, in accordance with a representative embodiment.

FIG. 3 illustrates a method for system for updating a medical facility, in accordance with a representative embodiment.

FIG. 4A illustrates another method for system for updating a medical facility, in accordance with a representative embodiment.

FIG. 4B illustrates another method for system for updating a medical facility, in accordance with a representative embodiment.

FIG. 4C illustrates another method for system for updating a medical facility, in accordance with a representative embodiment.

FIG. 5 illustrates a computer system, on which a method for system for updating a medical facility is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, a medical facility may be updated based on applying an artificial intelligence model to information obtained from the medical facility. Updating may include maintenance or upgrading, and may, for example include selective maintenance for equipment, selective upgrading for the medical facility, and/or selective upgrading for items of equipment at the medical facility. A system memory may store data from medical facilities including ICUs, and an AI-based algorithm may be used to suggest for updates for the medical facilities based on operational conditions and other information such as historical patient-flow with recovery status. Monitoring the operational status of medical facilities and proposing relevant upgrades may significantly enhance the ability of medical facilities to treat susceptible patients. Moreover, the teachings herein may improve overall life-support and services provided by the medical facilities.

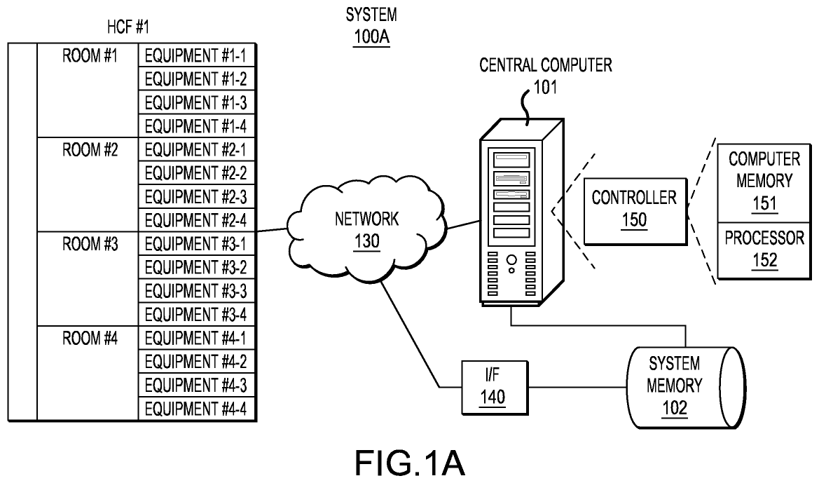

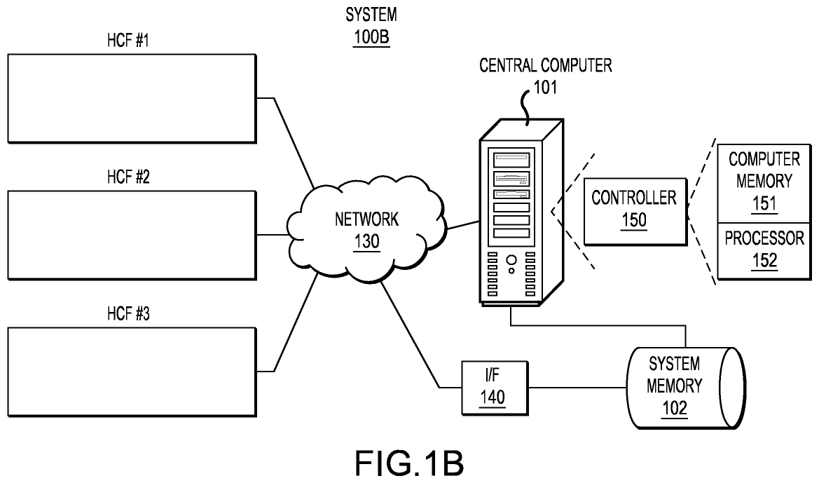

FIG. 1A illustrates a system for updating a medical facility, in accordance with a representative embodiment.

The system 100A in FIG. 1A is a system for system for updating a medical facility and includes components that may be provided together or that may be distributed. The system 100A includes a central computer 101, a system memory 102, a network 130, an interface 140 and a health care facility #1 (HCF #1). The central computer 101 includes a controller 150, and the controller 150 includes at least a computer memory 151 that stores instructions and a processor 152 that executes the instructions. The system 100A may be considered a centralized system insofar as the central computer 101 may be centralized and, in some embodiments, the interface 140 and the system memory 102 may be centralized with the central computer 101.

The HCF #1 is representative of health care facilities that store information such as for equipment and personnel in a memory. In FIG. 1A, the HFC #1 stores information for at least four rooms labelled as Room #1, Room #2, Room #3 and Room #4. The information for each of the four rooms is labelled as equipment #1-1, equipment #1-2, equipment #1-3 and equipment #1-4 for Room #1; equipment #2-1, equipment #2-2, equipment #2-3 and equipment #2-4 for Room #2; equipment #3-1, equipment #3-2, equipment #3-3 and equipment #3-4 for Room #3; equipment #4-1, equipment #4-2, equipment #4-3 and equipment #4-4 for Room #4. Of course, the HFC #1 or any other health care facility may include fewer or more than four rooms, and any particular room may include fewer or more than four items of equipment. The details of HFC #1 in FIG. 1A represent that various types of information may be maintained for any particular health care facility, and artificial intelligence as described herein may be applied to the various types of information to selectively determine whether and when to update the health care facility.

The central computer 101 may be provided at the health care facility, such as in an office, or may be provided remotely such as at a third-party service provider. The central computer 101 receives information from the HCF #1 over the network 130, such as via a local area network when the central computer 101 is local to the HCF #1 or over a wide area network when the central computer 101 is remote from the HCF #1.

The system memory 102 may also be provided at the health care facility, such as an in an office, or may be provided remotely such as at a third-party service provider. The system memory 102 is shown apart from the central computer 101 and from the HFC #1 in FIG. 1A because the system memory 102 may be provided together with or separate from the central computer 101 and the HFC #1. The system memory 102 may store large amounts of data for the HFC #1, including data on age and usage of the health care facility and the equipment at the health care facility, as well as on personnel at the health care facility such as data on which personnel use which items of equipment and how often.

The interface 140 may include a port, router, transmitter, receiver, transceiver or any other articular of manufacture that interfaces the system memory 102 with the network 103.

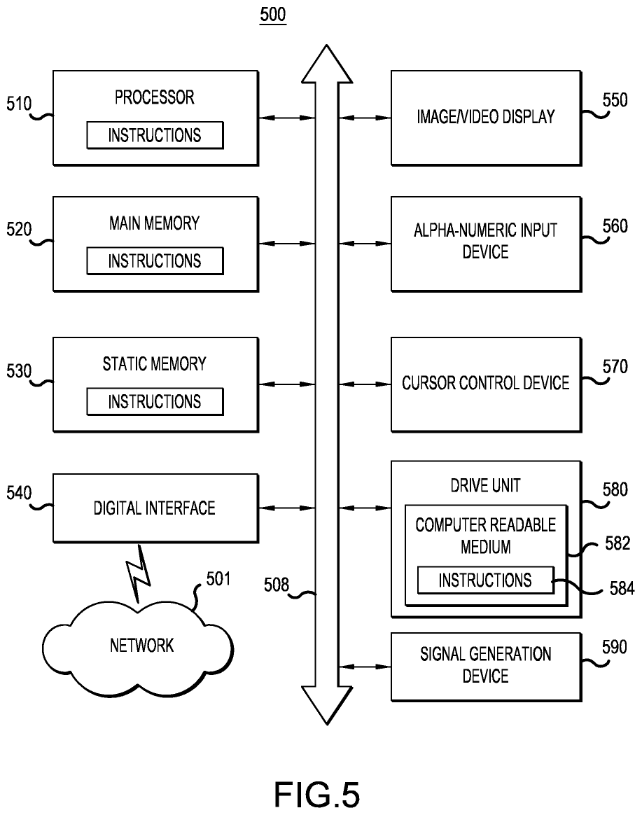

A computer that can be used to implement the central computer 101 is depicted in FIG. 5, though a central computer 101 may include more or fewer elements than depicted in FIG. 1 or FIG. 5.

Although not shown in FIG. 1A, a display may be provided local to the central computer 101 or remotely connected to the central computer 101. The display may be connected to the central computer 101 via a local wired interface such as an Ethernet cable or via a local wireless interface such as a Wi-Fi connection. The display may be interfaced with other user input devices by which users can input instructions, including mouses, keyboards, thumbwheels and so on. The display may be a monitor such as a computer monitor, a display on a mobile device, an augmented reality display, a television, an electronic whiteboard, or another screen configured to display electronic imagery. Such a display may be configured to display the data for the HCF #1 and other forms of displayable information consistent with the teachings herein.

The controller 150 may include interfaces, such as a first interface, a second interface, a third interface, and a fourth interface. One or more of the interfaces may include ports, disk drives, wireless antennas, or other types of receiver circuitry that connect the controller 150 to other electronic elements. One or more of the interfaces may also include user interfaces such as buttons, keys, a mouse, a microphone, a speaker, a display, or other elements that users can use to interact with the controller 150 such as to enter instructions and receive output.

The controller 150 may perform some of the operations described herein directly and may implement other operations described herein indirectly. For example, the controller 150 may indirectly control operations such as by retrieving information from the system memory 102. The controller 150 may directly control other operations such as logical operations performed by the processor 152 executing instructions from the computer memory 151 based on input received from electronic elements and/or users via the interfaces. Accordingly, the processes implemented by the controller 150 when the processor 152 executes instructions from the computer memory 151 may include steps not directly performed by the controller 150.

The controller 150 may implement an artificial intelligence model using the processor 152. For example, an artificial intelligence model may be implemented by one or more dedicated core(s) of a multi-core processor, or even a dedicated processor. Alternatively, the artificial intelligence model may be implemented by a processor that is also used to implement other operations besides applying the artificial intelligence model selectively to information retrieved from the system memory. The system memory 102 may obtain information from the HCF(s) periodically or continually so that the central computer 101 may maintain keen vigilance on the operational status of items of equipment in the HCF(s). The artificial intelligence model may be applied periodically or on-demand to monitor the comprehensive status of a HCF such as an ICU at a HCF, and suggest appropriate upgrades from time-to-time basis based on need and utility. The artificial intelligence model may suggest maintenance and upgrading of ICU resources. The artificial intelligence model may be trained from historical patient data, device usage data, and periodic device maintenance data, and generate appropriate upgrade triggers for better serviceability and advanced treatment. The artificial intelligence model may also encompass an operator management module which helps in specialized training of existing staffs and hiring of new medical facilitator for HCC/ICU support.

FIG. 1B illustrates another system for updating a medical facility, in accordance with a representative embodiment.

In FIG. 1B, the system 100B includes multiple HCFs compared to only the HCF #1 in FIG. 1A. That is, a central computer 101 may perform processes described herein for the HCF #1, the HCF #2, and the HCF #3, and the system memory 102 retrieves subsets of the data at the HCFs for the processes described herein. The multiple HCFs in FIG. 1B may be provided by a single entity or by different entities. The system 100B may be considered a centralized system insofar as the central computer 101 may be centralized and, in some embodiments, the interface 140 and the system memory 102 may be centralized with the central computer 101.

In FIG. 1A and FIG. 1B, the system memory 102 receives and stores information obtained from medical facilities via the interface 140. The interface 140 interfaces at least the system memory 102 to one or more communications networks such as the network 130. The central computer 101 is configured to retrieve from the system memory 102 the information obtained from the medical facility or medical facilities. The controller 150 is configured to apply an artificial intelligence model to the information obtained from each medical facility to determine whether to update the medical facility. The central computer 101 is configured to initiate at least one of maintenance of upgrading for the medical facility based on determining to update the medical facility.

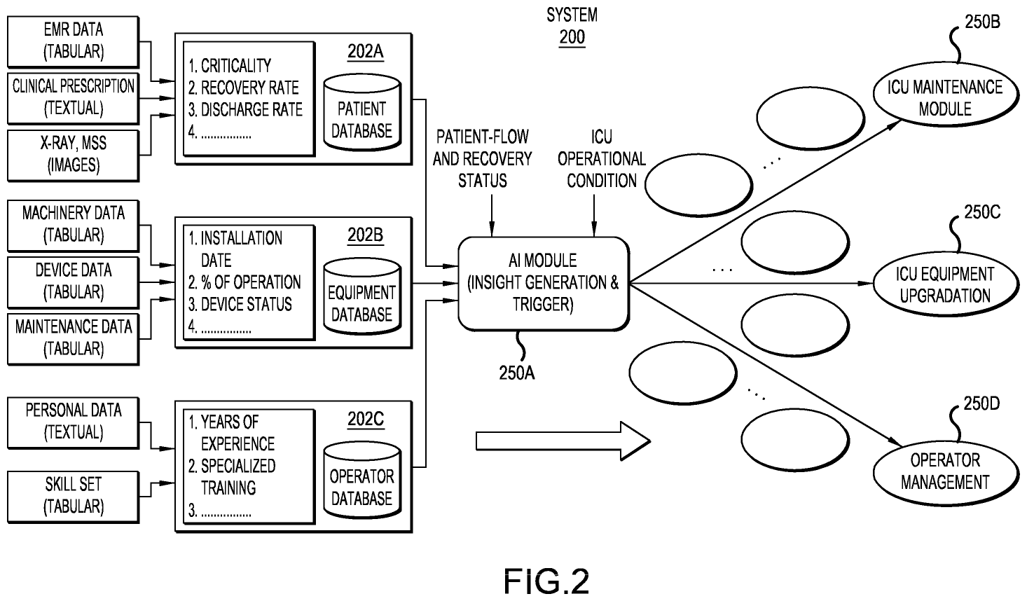

FIG. 2 illustrates an AI-based system for updating a medical facility, in accordance with a representative embodiment.

FIG. 2 is an AI-based system for suggesting maintenance and upgrading of ICU resources, as a particular implementation of the teachings herein. As shown, the system 200 includes a patient database 202A, an equipment database 202B, an operator database 202C, an AI module 250A, an ICU maintenance module 250B, an ICU equipment upgrade module 250C, and an operator management module 250D. The AI module 250A, the ICU maintenance module 250B, the ICU equipment upgrade module 250C, and the operator management module 250D may all be elements of a trained artificial intelligence model stored in the computer memory 151 and executed by the processor 152. The patient database 202A, the equipment database 202B and the operator database 202C may be implemented using the system memory 102 in FIG. 1A and in FIG. 1B. The AI module 250A, the ICU maintenance module 250B, the ICU equipment upgrade module 250C and the operator management module 250D may be implemented using the controller 150 in FIG. 1A and FIG. 1B. The system 200 may be considered a centralized system insofar as the elements of the system 200 may be centralized together, such as when implemented using the central computer 101 and the system memory 102.

The AI module 250A may include a data acquisition component and a core AI intelligence component. The data acquisition module of the AI module 250A may collect ICU-specific data such as information of various monitoring devices present and their respective condition, including how long the devices are in service, whether any irregularities have been observed, working status of the devices, and periodic maintenance history, etc. The data acquisition module of the AI module 250A may also amalgamate heterogeneous patient data such as image based: X-ray, MRI, etc.,; text-based: clinical prescriptions, order reports; and structured data into a centralized representation.

The core AI intelligence module of the AI module 250A may analyze historical ICU patient data to understand the patient inflow and the recovery during the stay. The core AI intelligence module of the AI module 250A may also integrate with pre/post ICU condition of the same patient monitoring data to evaluate the improvement in patient-condition for ICU support. The core AI intelligence module of the AI module 250A may learn the traits/characteristics of ICU-specific data and classify/rate them according to their current operational condition. The core AI intelligence module of the AI module 250A may identify the underperforming medical device based on current working condition based on the device working history, such as number of times the device needs to be restarted, number of times the device fails, etc.,) and the periodic maintenance history. The core AI intelligence module of the AI module 250A may also include a trigger module that determines whether to update the medical facility, such as by triggering under-performing ICU device performance alarms and communicating the same with the ICU maintenance module 250B. The core AI intelligence module of the AI module 250A may trigger under-performing and faulty device alarms and communicate with the ICU equipment upgrade module 250C. The core AI intelligence module of the AI module 250A may also triggers specialized upgrading of ICUs by analyzing the historical patient-data using demand assessment for critical diseases.

The AI module 250A may be responsible for insight generation utilizing historical patient and ICU data. The patient-specific data used as inputs to the artificial intelligence model implemented by the AI module 250A may include any one or more, for example, of patient clinical conditions, clinical reports, and/or tabular data for the ICU patients. Patient clinical conditions may include, for example, imaging data such as X-Ray and/or MRI-scans. Clinical reports may include, for example, prescriptions, lab reports or bills. Tabular data may include, for example, patient demographic data, vital measurements, or laboratory reports. The ICU data may involve specific details of a ICU such as how long the ICU is operational, types of operations supported by the ICU, details of the medical devices, history of maintenance, and more. The purpose of the AI module 250A is to integrate these heterogeneous datasets, generate meaningful information, and finally, provide suggestion for maintenance and upgrading of ICUs.

The AI module 250A may include a data acquisition and preprocessing component, and a core AI intelligence component. The data acquisition and preprocessing module may present the heterogenous data such as images, texts, and tabular data and provide a common framework to represent such heterogenous datasets in a machine-readable format. The data acquisition and preprocessing component may apply data-cleaning and formatting techniques to remove any duplicates and missing elements. The core AI intelligence component may produce a rank-based analysis while considering patient past records for various types of ICUs and their performance in critical time in terms of patient recovery time/status. From this rank-based system, the core AI intelligence component may generate triggers for under-performing and faulty medical devices in ICUs and forward the same to the ICU equipment upgrade module 250C and the ICU maintenance module 250B. Moreover, while analyzing patient data, if the AI module 250A detects mass casualty events such as many patients with dengue, the AI module 250A may generate triggers to upgrade multiple ICUs into a specific type such as ICUs for specific kind of diseases like dengue in order to support advanced treatments for the cause of the mass casualty event. The same kind of trigger may also be generated during outbreak of a disease which requires specialized ICU for therapy and cure.

The ICU maintenance module 250B may include software instructions stored in the computer memory 151 and executed by the processor 152. The ICU maintenance module 250B may contain information of various types of ICU devices and their maintenance status details, such as when they were last serviced or how long they have been without service. The ICU maintenance module 250B may be responsible for checking on the working status of triggered devices as alarmed by the AI module 250A, and determining if at all a maintenance is needed based on budget considerations, availability of maintenance staff, etc. The ICU maintenance module 250B may also automatically trigger monthly/annually maintenance of various ICUs and their respective monitoring devices. The ICU maintenance module 250B may also be responsible for integrating suggestions received from the AI module 250A with regular maintenance to reduce effective life-time cost of the ICU devices.

The ICU maintenance module 250B may be responsible for generating suggestions for maintenance requirement for various types of medical devices in ICUs. Different kinds of equipment may be required for different kinds of ICUs such as general medical, surgical, pediatric and neonatal ICUs. The ICU maintenance module 250B may be responsible for suggesting maintenance status details such as how long the ICU or equipment has gone without service. The ICU maintenance module 250B may also generate suggestions for maintenance of various devices such as cardiac monitors and/or mechanical ventilators. The ICU maintenance module 250B may be divided into two main sub-modules, including the regular maintenance sub-module and the trigger-based maintenance sub-module. The regular maintenance sub-module may be responsible for generating regular or scheduled maintenance suggestions. Different type of ICUs such as medical or surgical may have different schedule for maintenance, and may be highly depends on the type of severity of their operations. For example, maintenance for a surgical ICU responsible to monitor a recently operated heart-patient may be more critical than a medical ICU responsible for monitoring a patient whose sugar level has dropped and reported below margin. The schedules can be changed adaptively with the operational age of the ICUs and the types of patients being treated at the ICUs. For example, the ICUs that are in operational for a longer time may require more rapid maintenance than ICUs recently opened. The trigger-based maintenance sub-module may be responsible for analyzing the triggered maintenance request received from the AI module 250A and may validate the same while considering budget and other considerations. Budget considerations may be to minimize the lifetime cost of equipment. Other requirements may include availability of maintenance staff, routine staff schedules and more. The trigger-based maintenance sub-module may also generate suggestions to the user. Moreover, based on the above-mentioned factors, the trigger-based maintenance sub-module may be responsible for prioritizing a maintenance request before presenting the same to the user.

The ICU equipment upgrade module 250C may include software instructions stored in the computer memory 151 and executed by the processor 152. The ICU equipment upgrade module 250C may be responsible for generating suggestions for upgrading various ICU devices based on number of patient flow and type of patients, such as based on demographic information and/or the clinical conditions of the patients. The ICU equipment upgrade module 250C may also be responsible for inspecting the operational condition of triggered devices, once alerted by the AI module 250A, and determine on whether to repair/replace the faulty devices. The ICU equipment upgrade module 250C may also be responsible for suggesting acquisition of specialized and advanced equipment such as next generation devices to support specialized treatment including, for example, kidney failure, neurological trauma, and critical heart condition. The ICU equipment upgrade module 250C may also be responsible for proposing upgrades of existing ICUs for disease-specific or critical organ-support-based treatment. Moreover, the ICU equipment upgrade module 250C may be responsible for generating suggestions for specialized ICU upgrades considering budgetary requirements.

The ICU equipment upgrade module 250C may be responsible for suggesting any possible device upgrades for ICUs. The devices may be cardiac monitors which record electrical activity of heart, mechanical ventilators for artificial breathing support, BiPAP devices for respiratory support, suction machines for removing obstruction like blood or mucus, oxygen concentrators for maintaining blood oxygen level, infusion and syringe pumps for regulating medicine intake with titration. The ICU equipment upgrade module 250C may be helpful in identifying device status and inspecting the operational condition of the triggered devices once alerted by the AI module 250A. Finally, the ICU equipment upgrade module 250C may be determine whether to repair/replace the faulty devices, such as to remove a blockage present in the suction pipe or oxygen pipe or replace with new oxygen supply. The ICU equipment upgrade module 250C may be responsible for suggesting acquisition of specialized and advanced equipment such as next generation respiratory support for specialized treatment or advanced organ support (ADVOS) system for albumin dialysis procedures that can eliminate water-soluble and albumin-bound substances.

The ICU equipment upgrade module 250C may include two major upgrade sub-modules, including the device-specific upgrade sub-module and the ICU-specific sub-module. The device-specific upgrade sub module may collect the trigger request from the AI module 250A and verify the same, and finally, generate a suggestion to the user whether actual repair/replacement or upgrading is required for the ICU device. Additionally, the device-specific upgrade sub-module may validate other budgetary and staff-related constraints before finalizing and presenting a repair/replace request for the device. As an example, upgrading to next generation cardiac monitoring device may require an additional $5000 and special training for operating the device, whereas if requirements are not satisfied by the hospital staff or yearly budget, the device-specific upgrade sub-module may decide to discard the suggestion from the AI module 250A. The ICU-specific upgrade sub-module may also receive AI-based triggers for upgrading an ICU to support more-specialized treatment such as supporting an outbreak of COVID with respiratory assistance and advanced life-supporting machinery. Upgrading to such specialized ICUs may also require specialized training of staffs and hence, the ICU-specific upgrade sub-module may communicate the same with the operator management module 250D.

As set forth above, the ICU equipment upgrade module 250C may consider a variety of inputs including from patients flow and clinical condition of admitted patients for suggesting upgrades to and increases of medical devices.

The operator management module 250D may include software instructions stored in the computer memory 151 and executed by the processor 152. The operator management module 250D may be used to integrate operator data consisting of status and experience of ICU operators. The operator management module 250D may also be used to suggest acquiring/hiring new operators based on their expertise and demand/requirement(s) at the hospital and also upgraded ICU medical devices. The operator management module 250D may also be used to support specialized training facilitators, including selection of staff for specialized training using rank-based system and, after selecting the staffs, identifying type and number of trainings required while considering their current expertise/training and demand.

The operator management module 250D may be accountable for integrating operator data that contains the status and experience of ICU operators, current sets of available training and the list of trainings a specific operator completed, and types of specialized operators such as medical or surgery. The operator management module 250D may analyze any possible and necessary training upgrades available for any hospital operator and make suggestions accordingly. The operator management module 250D may also rank operators in terms of experience and schedule the operators accordingly for severe cases such as to ensure that the most critical patients are served with more skilled ICU operators. The operator management module 250D may also suggest hiring of and training for operators based on expertise, and demand/requirements at the medical facility. The operator management module 250D includes a training existing ICU operator sub-module, and a hiring new ICU operator sub-module. The training existing ICU operator sub-module may suggest required training for existing staff/workers and may also prioritize such training and generate corresponding schedules for training of each staff. As an example, the training existing ICU operator sub-module may schedule training related to handling next generation cardiac monitoring based on determining that this type of training is of utmost importance after acquiring such machinery. The training existing ICU operator sub-module may also consider training requirements for upgraded (new) medical devices. The hiring new ICU operator sub-module may be responsible for suggesting new hires for staff or medical facilitators related to ICUs. The hiring of a new hire may be as part of regular operations or may be triggered by a disease-outbreak. When hiring new staff, the training existing ICU operator sub-module may provide special priority to cases which require emergency attentions. For example, the training existing ICU operator sub-module may determine to hire COVID special staff with adequate training to set-up artificial respiratory system.

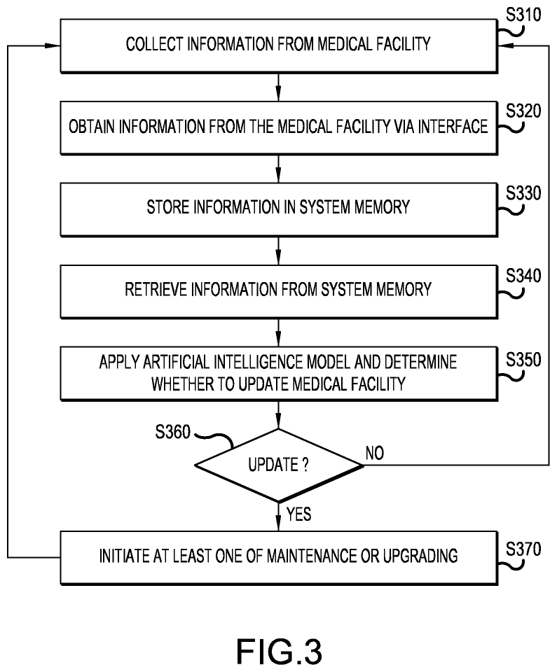

FIG. 3 illustrates a method for system for updating a medical facility, in accordance with a representative embodiment.

The method of FIG. 3 may be performed by the system 100A of FIG. 1A, the system 100B of FIG. 1B and/or the system 200 of FIG. 2.

The method of FIG. 3 may be performed mostly by a centralized system such as the combination of the central computer 101 and the system memory 102 in FIG. 1A and FIG. 1B. The method of starts at S310 by collecting information from a medical facility, such as at a health care facility. The medical facility may be the HCF #1 in FIG. 1A or any of the HCF #1, the HCF #2 or the HCF #3 in FIG. 1B, and may internally collect information via an information collection and archiving system. The collected information may be usage information of equipment and/or facilities, treatment information and corresponding demographic information of patients, and other types of information that may be valuable to an artificial intelligence model in terms of determining when to update any of the HCF #1, the HCF #2 or the HCF #3.

At S320, the information is obtained from the medical facility via an interface. For example, the information may be obtained via the network 130 and the interface 140 by the system memory 102. For example, the information may be periodically sent or retrieved, and/or may be sent or retrieved on-demand.

At S330, the information is stored in the system memory. For example, information sent to or retrieved by the system memory 102 may be stored.

At S340, information is retrieved from the system memory 102. For example, the central computer 101 may periodically retrieve specified information from the system memory 340, such as for a particular item or type of equipment or a particular facility.

At S350, an artificial intelligence model is applied to the information retrieved from the system memory 102. The artificial intelligence model is applied to determine whether to update the medical facility.

The controller 150 is used to implement an AI-based suggestive system for medical facilities, such as for ICU upgrades and the monitoring of ICU medical devices for periodic maintenance. The systems described herein are responsible for generating actionable insights associated with upgrades for medical facilities such as for ICUs, utilizing historical/real-time data while considering current working status of the medical facilities and metrics such as predicted patient inflow. The AI module 250A in FIG. 2 may be responsible for generating insights from historical data, whereas the ICU maintenance module 250B in FIG. 2 may be responsible for triggering routine maintenance, and the ICU equipment upgrade module 250C in FIG. 2 may be responsible for triggering acquisition of next generation equipment.

In some embodiments, the information applied to the artificial intelligence model may be information of patient conditions and recovery for one or more medical facilities. The information may be used to determine anomalies in terms of equipment usage, and patient outcomes in correlation with different staff members and different equipment. The information may also be applied from multiple facilities such as to compare outcomes and usage at the different facilities.

As one example of output from the artificial intelligence model at S350, the controller 150 may suggesting upgrades of the ICU components based on the equipment utilization and patient flow prediction. As another example, the controller 150 may suggest maintenance of ICU devices based on the equipment utilization and patient flow prediction. As yet another example, the controller 50 may manage device operators based on the device utilization history, such as by suggesting training or retraining

At S360, a determination is made whether to update the medical facility.

If the medical facility is to be updated (S360=Yes), at S370, at least one of maintenance or updating is initiated, and the process then returns to S310. If the medical facility is not to be updated (S360=No), the process returns to S310 without initiating an update.

The system 100A of FIG. 1A, the system 100B of FIG. 1B, the system 200 of FIG. 2, and the method of FIG. 3 may be used to avoid emergency conditions, wherein certain failures of medical devices in the ICU can be overcome with regular monitoring and maintenance. These systems and the method may also be used to preserve operational quality of the critical medical devices with systematic and methodical maintenance. Marginal errors in reporting health vitals such as blood pressure, pulse rate, oxygen level may be reduced with well-curated/monitored devices, insofar as this may result in more accurate assessments. These systems and the method may be used to upgrade to state-of-art machinery to improve vital estimation, disease detection, and health support to the patients. In extreme cases, such as during a disease outbreak, these systems and the method may be used to upgrade to a specialized ICU to assist in providing superior treatment and recovery. Keeping up with the recent trends with the development of devices can also assist in understanding any new pattern or irregularities in symptoms of a patient. Training the existing staff and recruiting specialized staff using these systems and the method may also aid in developing overall staff skill sets when devices are upgraded, and eventually improve the ICU operation and serviceability.

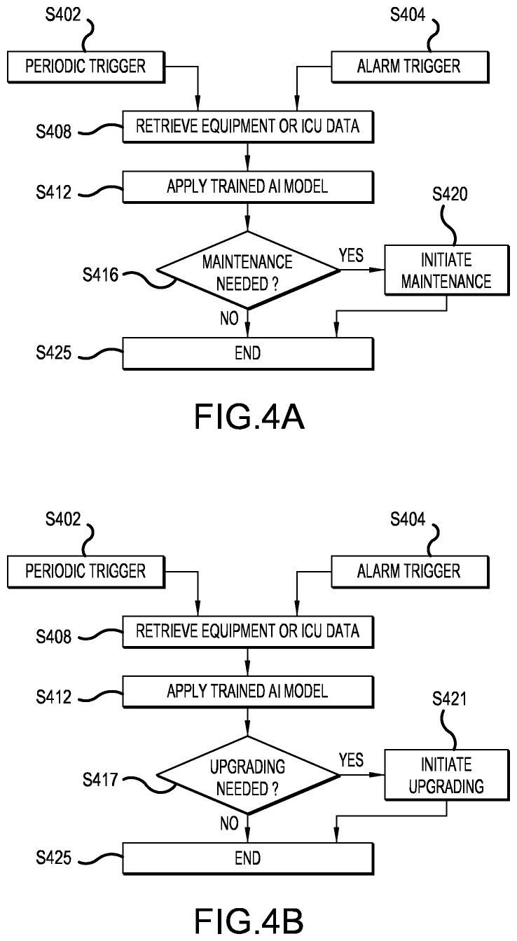

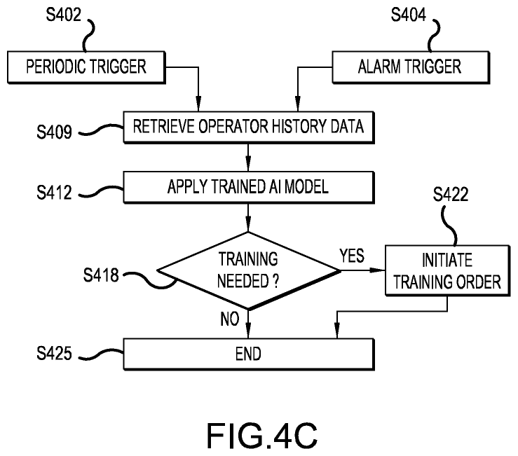

FIG. 4A illustrates another method for system for updating a medical facility, in accordance with a representative embodiment.

The method of FIG. 4A starts with either a periodic trigger at S402 or an alarm trigger at S404. A periodic trigger may be a timed trigger at a medical facility to perform automated collection of information, such as every 5 minutes, every 15 minutes, every hour, every 4 hours, every 6 hours, every 12 hours or every 24 hours. A medical facility may use multiple periodic triggers for different kinds of data collection, including multiple periodic triggers set at different timing intervals for different kinds of data. An alarm trigger may be a trigger generated by an alarm, such as when equipment at an ICU detects a malfunction or potential malfunction, or an notable event.

At S408, equipment or ICU data is retrieved. For example, data may be retrieved from equipment or from an information archiving system at a medical facility. The equipment data may be usage data for the amount of time equipment is used, the context in which the equipment is used, the movement of equipment between different rooms and areas of a medical facility, and patient data for patients on which the equipment is used. The ICU data may record movement of staff, patients and equipment into and out of the ICU.

At S412, a trained artificial intelligence model is applied to the data retrieved at S408. The trained artificial intelligence model may be trained to detect when various equipment should be maintained or upgraded, when various staff members should be hired or trained/retrained, when an ICU should be maintained or upgraded, and more. The training of the trained artificial intelligence model may be based on ground truths of timings of known failures of various items of equipment or staff, along with input information of the use and work history of the equipment and staff, such as from multiple different medical facilities.

At S416, a determination is made as to whether maintenance is needed. For example, the trained artificial intelligence model may determine that maintenance is needed for an item of equipment or for an ICU as a facility. If maintenance is needed (S416=Yes), maintenance is initiated at S420 and the process ends at S425. If maintenance is needed (S416=No), the process ends at S425 without initiating maintenance.

FIG. 4B illustrates another method for system for updating a medical facility, in accordance with a representative embodiment.

The method of FIG. 4B overlaps the method of FIG. 4A, except that the determination at S417 is for whether upgrading is required for equipment or the ICU, and at S421 if the answer is yes (S417=Yes), then upgrading is initiated.

FIG. 4C illustrates another method for system for updating a medical facility, in accordance with a representative embodiment.

The method of FIG. 4C overlaps the method of FIG. 4A and FIG. 4B, except at S409 operator history data is retrieved, at S418 the determination at S417 is for whether training is needed for an operator, and at S422 if the answer is yes (S418=Yes), then a training order is initiated.

As should be evident from the methods of FIG. 4A, FIG. 4B and FIG. 4C, one or more trained artificial intelligence models may be applied to data dynamically collected from medical facilities, and used to output recommendations for maintenance and upgrading. The types of maintenance and upgrading may be for equipment at the medical facilities, or for the medical facilities as a whole, as well as for staff employed at the medical facilities or who should be procured for employment at the medical facilities.

FIG. 5 illustrates a computer system, on which a method for system for updating a medical facility is implemented, in accordance with another representative embodiment.

Referring to FIG. 4, the computer system 500 includes a set of software instructions that can be executed to cause the computer system 500 to perform any of the methods or computer-based functions disclosed herein. The computer system 500 may operate as a standalone device or may be connected, for example, using a network 501, to other computer systems or peripheral devices. In embodiments, a computer system 500 performs logical processing based on digital signals received via an analog-to-digital converter.

In a networked deployment, the computer system 500 operates in the capacity of a server or as a client user computer in a server-client user network environment, or as a peer computer system in a peer-to-peer (or distributed) network environment. The computer system 500 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 500 can be incorporated as or in a device that in turn is in an integrated system that includes additional devices. In an embodiment, the computer system 500 can be implemented using electronic devices that provide voice, video or data communication. Further, while the computer system 500 is illustrated in the singular, the term “system” shall also be taken to include any collection of systems or sub-systems that individually or jointly execute a set, or multiple sets, of software instructions to perform one or more computer functions.

As illustrated in FIG. 5, the computer system 500 includes a processor 510. The processor 510 may be considered a representative example of a processor of a controller and executes instructions to implement some or all aspects of methods and processes described herein. The processor 510 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 510 is an article of manufacture and/or a machine component. The processor 510 is configured to execute software instructions to perform functions as described in the various embodiments herein. The processor 510 may be a general-purpose processor or may be part of an application specific integrated circuit (ASIC). The processor 510 may also be a microprocessor, a microcomputer, a processor chip, a controller, a microcontroller, a digital signal processor (DSP), a state machine, or a programmable logic device. The processor 510 may also be a logical circuit, including a programmable gate array (PGA), such as a field programmable gate array (FPGA), or another type of circuit that includes discrete gate and/or transistor logic. The processor 510 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 500 further includes a main memory 520 and a static memory 530, where memories in the computer system 500 communicate with each other and the processor 510 via a bus 508. Either or both of the main memory 520 and the static memory 530 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 520 and the static memory 530 are articles of manufacture and/or machine components. The main memory 520 and the static memory 530 are computer-readable mediums from which data and executable software instructions can be read by a computer (e.g., the processor 510). Each of the main memory 520 and the static memory 530 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 500 further includes a video display unit 550, such as a liquid crystal display (LCD), an organic light emitting diode (OLED), a flat panel display, a solid-state display, or a cathode ray tube (CRT), for example. Additionally, the computer system 500 includes an input device 560, such as a keyboard/virtual keyboard or touch-sensitive input screen or speech input with speech recognition, and a cursor control device 570, such as a mouse or touch-sensitive input screen or pad. The computer system 500 also optionally includes a disk drive unit 580, a signal generation device 590, such as a speaker or remote control, and/or a network interface device 540.

In an embodiment, as depicted in FIG. 5, the disk drive unit 580 includes a computer-readable medium 582 in which one or more sets of software instructions 584 (software) are embedded. The sets of software instructions 584 are read from the computer-readable medium 582 to be executed by the processor 510. Further, the software instructions 584, when executed by the processor 510, perform one or more steps of the methods and processes as described herein. In an embodiment, the software instructions 584 reside all or in part within the main memory 520, the static memory 530 and/or the processor 510 during execution by the computer system 500. Further, the computer-readable medium 582 may include software instructions 584 or receive and execute software instructions 584 responsive to a propagated signal, so that a device connected to a network 501 communicates voice, video or data over the network 501. The software instructions 584 may be transmitted or received over the network 501 via the network interface device 540.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, the system for updating a medical facility enables updating of a medical facility based on applying an artificial intelligence model to information obtained from the medical facility. Examples of the updating described above include, for example, selective maintenance for equipment, selective upgrading for the medical facility, and/or selective upgrading for items of equipment at the medical facility. Using the systems described herein when enhancing the functionality of a medical facility with advanced devices may assist in understanding any new patterns or irregularities in patient symptoms and thereby, result in proposals for more effective treatments. The systems described herein may also be used to train existing staff and recruit specialized staff to aid in developing overall skill sets, as this will improve ICU operations and serviceability in a facility.

Although system for updating a medical facility has been described with reference to several exemplary embodiments, it is understood that the words that have been used are words of description and illustration, rather than words of limitation. Changes may be made within the purview of the appended claims, as presently stated and as amended, without departing from the scope and spirit of system for updating a medical facility in its aspects. Although system for updating a medical facility has been described with reference to particular means, materials and embodiments, system for updating a medical facility is not intended to be limited to the particulars disclosed; rather system for updating a medical facility extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system for updating a medical facility, comprising:
a computer comprising a computer memory that stores instructions and a processor that executes the instructions;
an interface to a communications network, and
a system memory that receives and stores information obtained from the medical facility via the interface, wherein, based on the processor executing the instructions, the computer is configured to:
retrieve, from the system memory, the information obtained from the medical facility;
apply an artificial intelligence model to the information obtained from the medical facility to determine whether to update the medical facility; and
initiate at least one of maintenance or upgrading for the medical facility based on determining to update the medical facility.

2. The system of claim 1, wherein the system is a centralized system that is configured to determine whether to update a plurality of medical facilities including the medical facility and to initiate at least one of maintenance or upgrading for each of the plurality of medical facilities.

3. The system of claim 1, wherein the artificial intelligence model is configured to: determine whether to update the medical facility and determine whether to initiate maintenance for equipment in the medical facility.

4. The system of claim 1, wherein the artificial intelligence model is configured to: determine whether to update the medical facility and determine whether to initiate upgrading for the medical facility.

5. The system of claim 1, wherein: the system memory receives and stores usage information for a plurality of items of equipment at the medical facility, and the computer is configured to initiate at least one of maintenance or upgrading for each of the plurality of items of equipment at the medical facility.

6. The system of claim 1, wherein the computer is configured to intermittently retrieve, from the system memory, the information obtained from the medical facility; and intermittently apply the artificial intelligence model to the information obtained from the medical facility to determine whether to update the medical facility.

7. A method for updating a medical facility, comprising:
receiving and storing, at a system memory, information obtained from the medical facility via an interface;
retrieving, from the system memory by a system computer comprising a computer memory that stores instructions and a processor that executes the instructions, the information obtained from the medical facility;
applying, by the system computer, an artificial intelligence model to the information obtained from the medical facility to determine whether to update the medical facility; and
initiating, by the system computer, at least one of maintenance or upgrading for the medical facility based on determining to update the medical facility.

8. The method of claim 7, wherein the method is performed by a centralized system that is configured to determine whether to update a plurality of medical facilities including the medical facility and to initiate at least one of maintenance or upgrading for each of the plurality of medical facilities.

9. The method of claim 7, wherein the artificial intelligence model determines whether to update the medical facility and determines whether to initiate maintenance for equipment in the medical facility.

10. The method of claim 7, wherein the artificial intelligence model determines whether to update the medical facility and determines whether to initiate upgrading for the medical facility.

11. The method of claim 7, wherein: the system memory receives and stores usage information for a plurality of items of equipment at the medical facility, and the system computer is configured to initiate at least one of maintenance or upgrading for each of the plurality of items of equipment at the medical facility.

12. The method of claim 7, wherein the system computer is configured to intermittently retrieve, from the system memory, the information obtained from the medical facility; and intermittently apply the artificial intelligence model to the information obtained from the medical facility to determine whether to update the medical facility

13. A computer for updating a medical facility, comprising:
a computer memory that stores instructions; and
a processor that executes the instructions,
wherein, based on the processor executing the instructions, the processor is configured to:
retrieve, from a system memory that receives and stores information obtained from the medical facility via an interface, the information obtained from the medical facility;
apply an artificial intelligence model to the information obtained from the medical facility to determine whether to update the medical facility; and
initiate at least one of maintenance or upgrading for the medical facility based on determining to update the medical facility.

14. The computer of claim 13, wherein the computer is implemented in a centralized system that is configured to determine whether to update a plurality of medical facilities including the medical facility and to initiate at least one of maintenance or upgrading for each of the plurality of medical facilities.

15. The computer of claim 13, wherein the artificial intelligence model is configured to: determine whether to update the medical facility, determine whether to initiate maintenance for equipment in the medical facility, and determine whether to initiate upgrading for the medical facility.

16. The computer of claim 13, wherein:
the system memory receives and stores usage information for a plurality of items of equipment at the medical facility, and
the processor is further configured to initiate at least one of maintenance or upgrading for each of the plurality of items of equipment at the medical facility.

17. The computer of claim 13, wherein the processor is further configured to:
intermittently retrieve, from the system memory, the information obtained from the medical facility; and
intermittently apply the artificial intelligence model to the information obtained from the medical facility to determine whether to update the medical facility.
